(19) United States
(12) Patent Application Publication
(10) Pub. No.: US 2008/0213206 A1
(43) Pub. Date: Sep. 4, 2008

(54) Cosmetic Composition Comprising at Least One Fatty-Chain Polylysine, Which is Intended to Improve the Surface State of Keratin Fibers

(76) Inventors: Michel Philippe, Wissous (FR); Alexandra Metois, Athis Mons (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/997,922

(22) PCT Filed: Aug. 3, 2006
(86) PCT No.: PCT/FR2006/050780

§ 371 Date: Feb. 5, 2008

(30) Foreign Application Priority Data
Aug. 5, 2005 (FR) 0552449

(51) Int. Cl. A61K 8/40; A61Q 5/00

(52) U.S. Cl. 424/70.1

(57) ABSTRACT

The present invention relates to a cosmetic composition comprising, in a cosmetically acceptable medium, at least one fatty-chain polylysine. The composition of the invention enables to improve the surface state of keratin fibres. The invention also relates to methods for producing fatty-chain polylysines, as well as new compounds.

The present invention relates to new cosmetic compositions, comprising at least one fatty-chain polylysine, which are intended to improve the surface state of keratin fibres, as well as synthetic methods for producing fatty-chain polylysines.

Many cosmetic compositions which are intended to protect or to repair a damaged hair are known. However, such compositions give vary different results that are sometimes insufficient. It thus appears to be necessary to develop new compositions to improve the surface state of keratin fibres.

Using fatty-chain N-ε-polylysines (amide junction) was described in the patent application JP 0 5246 963, in the food industry as a bacterial emulsifier. Using fatty-chain N-ε-polylysines (amide junction) was also described in the patent application JP/2001-191521, as a bactericide detergent. Urethane analogues were described in the patent application JP/2002-147360. Lastly, preparing amide function derivatives was also described, as well as their advantage as surfactants, in the patent application JP 1 1255 892.

The applicant surprisingly just discovered that a composition comprising at least one fatty-chain polylysine makes it possible to improve the surface state of keratinic materials.

It is thus an object of the invention to provide a cosmetic composition intended to protect and/or to repair the keratinic materials, characterized in that it comprises, in a cosmetically acceptable medium, at least

one fatty-chain polylysine of following formula (I):

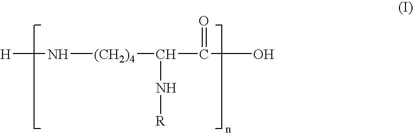

as well as salts and isomers thereof;
(a) wherein R represents H or a moiety of either of following formulas (II), (III), (IV) or (V):

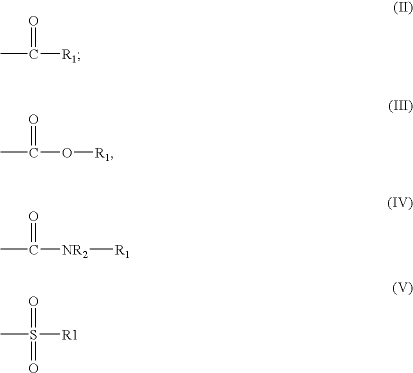

and,
wherein R₁ represents a linear or branched, saturated or unsaturated, C5 to C30 hydrocarbon chain, optionally interrupted with N, S, or O, and optionally substituted with one or more hydroxyl group(s) and/or one or more fluorine atom(s), and R₂ represents H, or a linear or branched, saturated or unsaturated C1 to C8 alkyl group, and
(b) n is from 2 to 10000 and preferably from 5 to 1000,
and under the proviso that in the polylysine of formula (I) at least one of the R radicals be different from H.

As used herein, a keratinic material is intended to mean skin appendages and especially hair, eyelashes and nails Preferably, it here refers to a human keratinic material and preferably to hair.

The hereabove described cosmetic composition makes it possible to provide a damaged keratinic material that has become hydrophilic with a hydrophobic coating or sheathing. Such hydrophobic sheathing enables the damaged fibre to durably recover a hydrophobic surface state approaching that of natural hair. For example, the hair appearance looks better when treated with a cosmetic composition of the invention. The hair treated with the composition of the invention also feels smoother, is more shiny, easier to detangle and to style.

According to a preferred embodiment, in the fatty-chain polylysine of formula (I), the R radical represents H or a moiety of either of following formulas (II) or (IV) such as previously defined wherein R1 represents a linear or branched, saturated or unsaturated C5 to C24 hydrocarbon chain and R2 represents a hydrogen atom, being understood that at least one of the R radicals is different from H.

Preferably, in the fatty-chain polylysine of formula (I), the R radical represents H or a moiety of either of following formulas (VI), (VII), (VIII) or (IX):

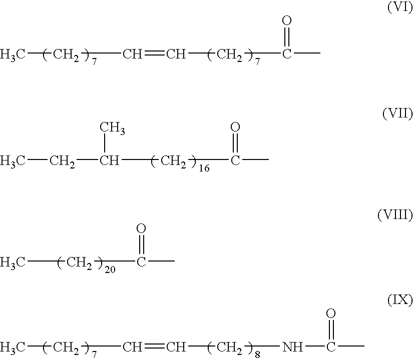

being understood that at least one of the R radicals is different from H.

The present invention also relates to organic or inorganic salts of compounds (I), where the lysine unit may moreover be of the racemic or isomerically pure type.

Generally speaking, the organic or inorganic salts to be suitably used according to the invention are the physiologically acceptable salts of compounds of formula (I).

The addition salts of an acid are for example salts of hydrochloric acid or hydrobromic acid or sulphuric or citric or succinic acid or tartaric or lactic acid or paratoluene sulfonic or phosphoric or acetic acid, or salts of fatty acids such as linoleic, oleic, palmitic, stearic, behenic and 18-methyleicosanoic acids.

The addition salts of a base are for example sodium and calcium salts, and hydroxyalkyl amine salts such as for example N-methyl glucamine or aminopropane diol.

It is a further object of the present invention to provide new compounds having general formula (X):

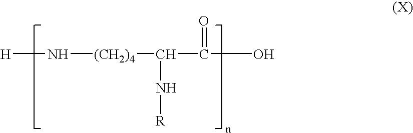

as well as salts or isomers thereof;

(a) wherein R represents H or a moiety of either of following formulas (IV) or (V):

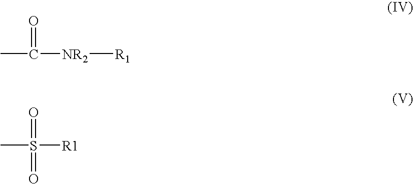

and,
wherein R₁ represents a linear or branched, saturated or unsaturated C5 to C₃₀ hydrocarbon chain, optionally interrupted with N, S, or O, and optionally substituted with one or more hydroxyl group(s) and/or one or more fluorine atom(s), and R₂ represents H, or a linear or branched, saturated or unsaturated C1 to C8 alkyl group, and
(b) n is from 2 to 10000 and preferably from 5 to 1000,
under the proviso that in the polylysine of formula (x) at least one of the R radicals be different from H.

According to a particular embodiment, in the fatty-chain polylysine of formula (X), the R radical represents H or a moiety of either of following formulas (IV) or (V) such as previously defined, wherein R1 represents a linear or branched, saturated or unsaturated C5 to C24 hydrocarbon chain and R2 represents a hydrogen atom, being understood that at least one of the R radicals is different from H.

More preferably, in the fatty-chain polylysine of formula (X), the R radical represents a radical of formula (IV) wherein R2 represents a hydrogen atom and R1 represents a CH3—(CH2)₇—CH═CH—(CH2)₈— chain.

The cosmetic composition of the invention may comprise from 0.01 to 50%, and preferably from 0.05 to 20% by weight, of fatty-chain polylysine, as related to the composition total weight.

The cosmetic compositions of the invention comprise, in addition to said compounds of formula (I), a physiologically acceptable medium, and particularly a cosmetically acceptable medium, that is to say a medium which is compatible with the keratinic materials as previously defined.

As an example, said physiologically acceptable medium may comprise various components traditionally used for treating keratin fibres such as hair, amongst which water or organic solvents may be mentioned, such as alcohols like ethanol or benzyl alcohol, or fragrances, preserving agents, sequestering agents, acidifying agents, alkalinizing agents, UV absorbing filters, pigments, pearlescent agents, fillers, colouring agents, polymers different from compounds of formula (I), thickeners, anionic or amphoteric or non ionic surfactants, emulsifying agents, coemulsifying agents, gums, conditioning agents, trace elements, agents for combating hair loss, anti-dandruff agents, ceramides, agents for hair reshaping, waxes, polar or apolar mineral, organic or vegetable oils, volatile or non volatile silicones, vitamins or provitamins, anti-free radical agents, antioxidants, oxidants as well as their combinations.

Of course the man skilled in the art will be able to choose this or these optional component(s) and/or in which amount, so that the advantageous properties of the composition of the invention will not be affected, or substantially not.

According to a particular embodiment, the cosmetically acceptable medium containing the fatty-chain polylysines of the invention comprises:
water, and/or
aliphatic or aromatic alcohols, preferably ethanol, benzyl alcohol, fatty alcohols, polyols that have been modified or not, such as glycerol, glycol, propylene glycol, dipropylene glycol, butylene glycol, butyl diglycol, and/or
volatile or non volatile silicones, and/or
mineral, organic or vegetable oils, and/or
oxyethylenated or non oxyethylenated waxes, paraffins, alkanes and preferably C₅ to C₁₀ alkanes, and/or
fatty acids, fatty amines, fatty esters and more particularly fatty alcohol benzoates or salicylates, and/or
acetone, methyl ethyl ketone, methyl acetate, butyl acetate, ethyl acetate, dimethoxyethane, diethoxyethane,
and mixtures thereof.

The pH of the composition of the present invention may be adjusted to the expected value by means of acidifying or alkalinizing agents that are well known from the state of the art.

Suitable examples of alkalinizing agents include ammonia, alkaline carbonates, alkanol amines such as mono-, di- and triethanol amines as well as their derivatives, hydroxyalkyl amines and oxyethylenated and/or oxypropylenated ethylene diamines, sodium or potassium hydroxides and compounds of following formula (XI):

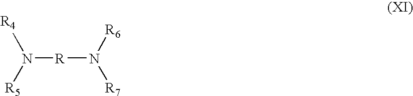

wherein R is a propylene residue optionally substituted with a hydroxyl moiety or a C₁-C₄ alkyl radical; R₄, R₅, R₆ and R₇, being the same or different, represent a hydrogen atom, a C₁-C₄ alkyl radical or a C₁-C₄ hydroxyalkyl radical.

Suitable examples of acidifying agents traditionally include inorganic or organic acids such as hydrochloric acid, orthophosphoric acid, carboxylic acids such as tartaric acid, citric, lactic acid, or sulfonic acids.

The cosmetically acceptable medium may present as such or be in an emulsified or encapsulated form.

The hereabove described cosmetic composition may be rinsed off or not, and may present in various galenic forms.

In particular, the cosmetic composition may be a composition to be applied onto the hair, coming as a lotion, a spray (aerosol or not), a foam, a gel, a cream, a stick. This composition may be a shampoo, an after-shampoo, a temporary or a permanent hair colour composition or a hair reshaping composition such as for perming or defrizzing or straightening the hair.

It is a further object of the invention to provide synthetic methods for producing fatty-chain polylysines of formula (I), such as described hereunder.

Thus, the present invention relates to a synthetic method for producing a fatty-chain polylysine of following formula (I) consisting in reacting:
(i) a compound of formula:

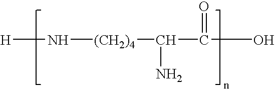

with
(ii) a compound selected from:
   a) a compound of formula:

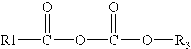

wherein R₁ represents a linear or branched, saturated or unsaturated C5 to C30 hydrocarbon chain, optionally interrupted with N, S, or O, and optionally substituted with one or more hydroxyl group(s) and/or one or more fluorine atom(s), and R₃ represents a linear or branched, saturated or unsaturated C1 to C8 alkyl group,
   to obtain compounds of formula (II) such as previously defined;
b) a compound of formula:

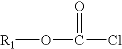

wherein R1 is such as defined in a)
   to obtain compounds of formula (III) such as previously defined;
c) a compound of formula:

R₁—N═C═O
wherein R1 is such as defined in a)
   to obtain compounds of formula (IV) such as previously defined for which R2=H;
d) a compound of formula:

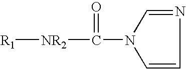

wherein R1 is such as defined in a)
and R2 represents H, or a linear or branched,
saturated or unsaturated C1 to C8 alkyl group,
   to obtain compounds of formula (IV) such as previously defined,
e) a compound of formula:

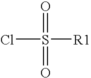

wherein R1 is such as defined in a)
   to obtain compounds of formula (V) such as previously defined.

### General Procedure for Grafting N-ε-Poly-Lysine:

N-ε-Polylysine was solubilized between 20 and 80° C. in a protic solvent such as alcohols. The activated species (compound of formula as previously defined in a, b, c, d or e) in solution in an organic solvent such as THF was added dropwise to the reaction medium, and allowed to remain under stirring at room temperature for 1H to 48H; the solution being then either concentrated to dryness or concentrated and extracted with water to recover the compound of formula (I). Washing several times the resulting powder with an organic solvent could then be optionally conducted.

The present invention will be more readily understood thanks to the following illustrative examples without in any case being limited thereto.

### EXAMPLES

### Example 1

### Synthesizing a Fatty-Chain Polylysine of Following Formula (I), Comprising at Least One R Radical of Following Formula

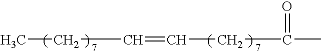

### Synthesizing the Mixed Anhydride

0.74 g of oleic acid (tech.) and 0.24 g of triethyl amine were diluted in 5 ml THF. This solution was added, by very slowly dropping, to a solution of 0.27 g of ethyl chloroformate and 2 ml of THF, cooled to −10° C. The addition was made such that the temperature be ranging from −10° C. to −5° C. The thus produced triethyl amine hydrochloride did instantaneously precipitate. Once the addition was completed, the mixture was allowed to come back to room temperature, then the solution was filtered. The mixed anhydride-containing filtrate was immediately used as such in the next step.

### Grafting the Mixed Anhydride

10 g of N-ε-Poly-L-lysine (Chisso) were solubilized at 60° C. in 100 ml of 96% ethanol. The mixture was allowed to come back to room temperature. As soon as the mixed anhydride preparation was complete, the solution containing it was added dropwise thereto. Once the addition was complete, the mixture was stirred for 15 minutes. The reaction medium was then concentrated, then diluted in 90 ml of water. The free residual acid was extracted 3 times with 300 ml of ethyl acetate. The aqueous phase was then evaporated to dryness. The resulting compound was characterized by 2D NMR, GC and functional assays. It did correspond to the expected product.

Resulting weight: 7 g. Appearance: yellow powder. Yield: 65% Grafting rate: 1 mole of graft chain per mole of N-ε-Poly-L-Lysine.

The polylysine fatty chain rate may be adjusted by varying the molar ratio [mixed anhydride]/[N-ε-Poly-L-lysine].

Polylysines having a behenic acid chain and polylysines having a 18-methyleicosanoic acid chain may be synthesized according to the same procedure.

### Example 2

### Synthesizing a Fatty-Chain Polylysine of Following Formula (I), Comprising at Least One R Radical of Following Formula

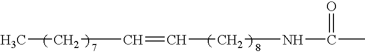

0.6 g of oleyl amine (2.36 mmol_{—}1 eq) in a solution in 10 ml of CH₂Cl₂ were added dropwise to 0.47 g of carbonyl diimidazole (2.88 mmol_{—}1.22 eq) in a solution in 5 ml of CH₂Cl₂. The carbonyl diimidazole removal was monitored by a thin-layer chromatography on SiO₂ (eluent: 90/10 CH₂Cl₂/MeOH).

This reaction medium was added dropwise to a solution containing 10 g of anhydrous N-ε-Poly-L-lysine (Chisso) (2.13 mmol_{—}0.9 eq) in 100 ml 95% EtOH; the resulting medium was then stirred at room temperature for 48 hours.

The reaction medium was then concentrated to dryness on a rotary evaporator under reduced pressure. The pale yellow powder obtained was suspended in 100 ml CH₂Cl₂ and refluxed for 1H30, then filtered. The operation was repeated twice. The end product was obtained as a pale yellow powder (9.69 g) with a yield of 90%.

The grafting was confirmed by 2D NMR (¹H-¹³C) and measured (1 mole of graft fatty chain per mole of N-ε-poly-L-Lysine) by ¹H NMR.

¹H NMR (400 MHz, D₂O) δ (ppm): 0.758 (m), 1.07 (m), 1.43 (m), 1.53 (m), 2.83 (t), 3.11 (m), 3.22 (t), 3.44 (m), 3.53 (q), 7.01 (s), 7.66 (s)

Examples of cosmetic compositions:

### Example 3

### Shampoo-Resistant Brushing Composition

| | | |
|---|---|---|
| Compound of example 1 | 10 | g |
| Water | qs 100 | g |

The lotion was sprayed onto wet towel-dried hair after rinsing the shampoo.

The hair was combed through after spraying the lotion and brushing was directly carried out.

Following this procedure makes the hair easier to style. The hair straightening is more pronounced and resists to shampoo.

### Example 4

Shampoo-resistant brushing composition

| | | |
|---|---|---|
| Compound of example 1 | 8 | g |
| Ethanol | 2 | g |
| Dow Corning 245 Fluid | 9 | g |
| Dow Corning 1501 Fluid | 9 | g |
| Water | qs 100 | g |

The lotion was sprayed onto wet towel-dried hair after rinsing the shampoo.

The hair was combed through after spraying the lotion and brushing was directly carried out.

Following this procedure makes the hair easier to style. The hair straightening is more pronounced and resists to shampoo.

### Examples 5 to 10

### Shampoos

Shampoo Compositions were Prepared as Follows:

| Ingredients | Ex. 5 | | Ex. 6 | | Ex. 7 | | Ex. 8 | | Ex. 9 | | Ex. 10 | |
|---|---|---|---|---|---|---|---|---|---|---|---|---|
| SODIUM LAURETH SULFATE [1] | 12% | am | 12% | am | 14% | am | 14% | am | 14% | am | 14% | am |
| COCO-BETAINE [2] | — | | — | | — | | — | | 2% | am | 2% | am |
| COCAMIDOPROPYL BETAINE [3] | 2% | am | 2% | am | 3% | am | 3% | am | — | | — | |
| POLYQUATERNIUM 10 [4] | — | | 0.2% | am | | | 0.3% | am | — | | — | |
| Compound of example 1 | 0.5% | am | 0.5% | am | 0.5% | am | 0.5% | am | 0.5% | am | 0.5% | am |
| GUAR | — | | — | | — | | — | | — | | 0.1% | am |
| HYDROXYPROPYLTRIMONIUM | | | | | | | | | | | | |
| CHLORIDE [6] | | | | | | | | | | | | |
| DIMETHICONE [7] | — | | — | | — | | — | | 3% | am | 3% | am |
| DIMETHICONE [8] | — | | — | | 2% | am | 2% | am | — | | — | |
| COCAMIDE MIPA [9] | — | | — | | 1.5% | am | 1.5% | am | 0.5% | am | 0.5% | am |
| NaCl | 1% | am | 1% | am | 0.5% | am | 0.5% | am | 0.5% | am | 0.5% | am |

| | | | | | | |
|---|---|---|---|---|---|---|
| PRESERVING AGENT | Qs | Qs | Qs | Qs | Qs | Qs |
| FRAGRANCE | Qs | Qs | Qs | Qs | Qs | Qs |
| SODA/CITRIC ACID | Qs pH 6 | Qs pH 7 | Qs pH 7 | Qs pH 7 | Qs pH 7 | Qs pH 7 |
| WATER | QsP 100 | QsP 100 | QsP 100 | QsP 100 | QsP 100 | QsP 100 |

[1] Texapon N 702 (Cognis)
[2] Dehyton AB 30 (Cognis)
[3] Tego betaine F50 (Goldschmidt)
[4] Ucar polymer JR400 LT (Amerchol)
[5] Compound of example 1
[6] Jaguar C13S (Rhodia)
[7] Belsil DM 300 000 (Wacker)
[8] Mirasil DM 500 000 (Rhodia)
[9] Empilan CIS (Huntsman)

The hair was shampooed with one of the compositions of examples 5 to 10, then rinsed with water. The thus treated hair exhibits excellent cosmetic properties (softness, natural feel).

### Examples 11 to 14

### After-Shampoos

The after-shampoo compositions were prepared as follows:

| Ingredients | Ex. 11 | Ex. 12 | Ex. 13 | Ex. 14 |
|---|---|---|---|---|
| CETEARYL ALCOHOL [10] | 3% am | 3% am | 3% am | 3% am |
| CETYL ESTERS [11] | 1% am | 1% am | 1% am | 1% am |
| BEHENTRIMONIUM CHLORIDE [12] | — | — | 2% am | 2% am |
| AMODIMETHICONE [13] | — | 1% am | — | 1% am |
| OLEIC ACID CHAIN POLYLYSINE [7] | 1% am | 1% am | 1% am | 1% am |
| PRESERVING AGENT | Qs | Qs | Qs | Qs |
| FRAGRANCE | Qs | Qs | Qs | Qs |
| WATER | QsP 100 | QsP 100 | QsP 100 | QsP 100 |

[7] Belsil DM 300 000 (Wacker)
[10] Lanette O OR (CognisS)
[11] Miraceti (Laserson)
[12] Genamin KDMP (Clariant)
[13] Dow Corning 939 emulsion (Dow corning)

The hair was shampooed and rinsed with water. One of the compositions of examples 11 to 14 was then applied onto the hair, and thereafter rinsed with water. The thus treated hair exhibits excellent cosmetic properties (softness, natural feel).

1. A cosmetic composition intended to protect and/or to repair the keratinic materials, characterized in that it comprises, in a cosmetically acceptable medium, at least one fatty-chain polylysine of following formula (I): 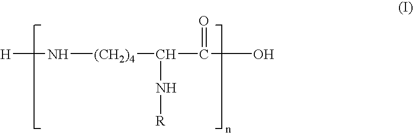
(a) wherein R represents H or a moiety of either of following formulas (II), (III) (IV) or (V): 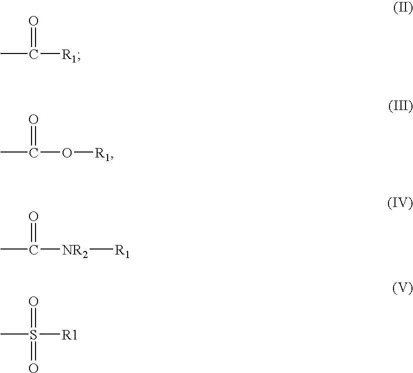
and,
wherein R₁ represents a linear or branched, saturated or unsaturated C5 to C30 hydrocarbon chain, optionally interrupted with N, S, or O, and optionally substituted with one or more hydroxyl group(s) and/or one or more fluorine atom(s), and R₂ represents H, or a linear or branched, saturated or unsaturated C1 to C8 alkyl group, and
(b) n is from 2 to 10000 and preferably from 5 to 1000;
as well as salts and isomers thereof;
under the proviso that in the polylysine of formula (I) at least one of the R radicals be different from H.

2. A cosmetic composition according to claim 1, wherein the R moiety is selected from moieties of formula (II) or (IV).

3. A cosmetic composition according to any preceding claim, wherein R1 represents a linear or branched, saturated or unsaturated C5 to C24 hydrocarbon chain and R2 represents a hydrogen atom.

4. A cosmetic composition according to any of claims 1 to 3, wherein the R radical represents a moiety of either of following formulas (VI), (VII), (VIII) or (IX):

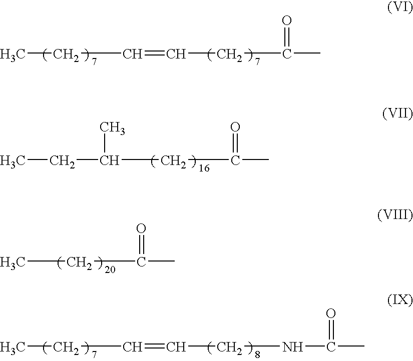

5. A cosmetic composition according to any of claims 1 to 4, which comprises from 0.01 to 50%, and preferably from 0.05 to 20% by weight, of fatty-chain polylysine of formula (I), as related to the composition total weight.

6. A cosmetic composition according to any of claims 1 to 5, wherein the cosmetically acceptable medium comprises:
water, and/or
aliphatic or aromatic alcohols, preferably ethanol, benzyl alcohol, fatty alcohols, polyols that have been modified or not, such as glycerol, glycol, propylene glycol, dipropylene glycol, butylene glycol, butyl diglycol, and/or
volatile or non volatile silicones, and/or
mineral, organic or vegetable oils, and/or
oxyethylenated or non oxyethylenated waxes, paraffins, alkanes and preferably C₅ to C₁₀ alkanes, and/or
fatty acids, fatty amines, fatty esters and more particularly fatty alcohol benzoates or salicylates, and/or
acetone, methyl ethyl ketone, methyl acetate, butyl acetate, ethyl acetate, dimethoxyethane, diethoxyethane,
and mixtures thereof.

7. A cosmetic composition according to any of claims 1 to 6, wherein the cosmetically acceptable medium presents as such or in an emulsified or encapsulated form.

8. A cosmetic composition according to any of claims 1 to 7, which comprises one or more volatile or non volatile silicones.

9. A cosmetic composition according to any of claims 1 to 8, which comprises a hair fibre protecting agent selected from the group consisting of UV absorbing filters, anti-free radical agents, antioxidants, vitamins, provitamins, as well as mixtures thereof.

10. A cosmetic composition according to any of claims 1 to 9, which composition is intended to be applied onto the hair and comes as a lotion, a spray (aerosol or not), a foam, a gel, a cream, a stick, a shampoo, an after-shampoo, a temporary or a permanent hair colour composition or a hair reshaping composition such as for perming or defrizzing or straightening the hair.

11. A compound having general formula (X): 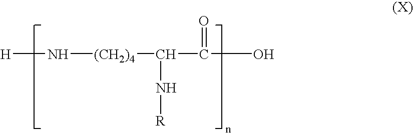
as well as salts and isomers thereof;
(a) wherein R represents H or a moiety of either of following formulas (IV) or (V): 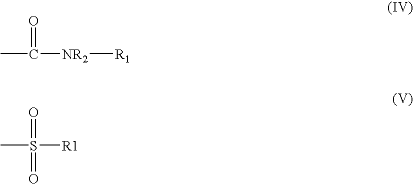
and,
wherein R₁ represents a linear or branched, saturated or unsaturated C5 to C30 hydrocarbon chain, optionally interrupted with N, S, or O, and optionally substituted with one or more hydroxyl group(s) and/or one or more fluorine atom(s), and R₂ represents H, or a linear or branched, saturated or unsaturated C1 to C8 alkyl group, and
(b) n is from 2 to 10000 and preferably from 5 to 1000,
under the proviso that in the polylysine of formula (x) at least one of the R radicals be different from H.

12. A compound according to claim 11, wherein the R radical represents H or a moiety of either of following formulas (IV) or (V) such as previously defined, wherein R1 represents a linear or branched, saturated or unsaturated C5 to C24 hydrocarbon chain and R2 represents a hydrogen atom, being understood that at least one of the R radicals is different from H.

13. A compound according to claim 11 or 12, wherein the R radical represents a radical of formula (IV) wherein R2 represents a hydrogen atom and R1 represents a CH3—(CH2)₇—CH═CH—(CH2)₈-chain.

14. A synthetic method for producing a fatty-chain polylysine of formula (I),
characterized in that it consists in reacting:
(i) a compound of formula: 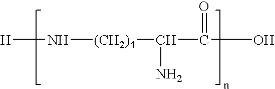
with
(iii) a compound selected from:
a) a compound of formula: 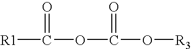
wherein R₁ represents a linear or branched, saturated or unsaturated, C₅ to C₃₀ hydrocarbon chain, optionally interrupted with N, S, or O, and optionally substituted with one or more hydroxyl group(s) and/or one or more fluorine atom(s), and R₂ represents H, or a linear or branched, saturated or unsaturated C1 to C8 alkyl group;
b) a compound of formula: 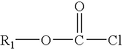
wherein R1 is such as defined in a);
c) a compound of formula:
R₁N═C═O
wherein R1 is such as defined in a);
d) a compound of formula: 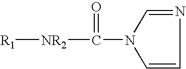
wherein R1 is such as defined in a); and R2 represents H, or a linear or branched, saturated or unsaturated C1 to C8 alkyl group;
e) a compound of formula: 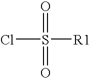
wherein R1 is such as defined in a).

15. Use of a cosmetic composition according to any of claims 1 to 10 for treating keratinic materials.

16. Use according to claim 15 for improving the surface state of keratinic materials.
